(19) United States
(12) Patent Application Publication
(10) Pub. No.: US 2004/0115226 A1
(43) Pub. Date: Jun. 17, 2004

(54) Free-flowing solid formulations with improved bio-availability of poorly water soluble drugs and process for making the same

(76) Inventors: Wenji Li, Aliso Viejo, CA (US); Edward Alosio, Coto De Caza, CA (US); Bricini Faith (Bim) Dema-Ala, Aliso Viejo, CA (US); Amy Nguyen, Newport Beach, CA (US)

Correspondence Address: Gabor L. Szekeres, Suite 112, 8141 E. Kaiser Boulevard, Anaheim Hills, CA, 92808, US

(21) Appl. No.: 10/317,657

(22) Filed: Dec. 12, 2002

(51) Int. Cl. A61K 9/00; A61K 9/127

(52) U.S. Cl. 424/400; 424/450

(57) ABSTRACT

A free-flowing solid formulations of drugs or pharmaceutical agents which have poor aqueous solubility are obtained by admixing a liquid or gel composition that includes 1 to 30 per cent by weight of the drug, 5 to 60 per cent by weight of a surfactant, 10 to 40 per cent by weight of water; 1 to 20 per cent by weight of unsaturated fatty acid ester, 0 to 50 per cent by weight water miscible pharmaceutically acceptable polyol and 1 to 10 per cent by weight of phospholipid with a pharmaceutically acceptable suitable solid carrier and thereafter drying the admixture. The free-flowing powder is suitable for being formed into tablets or capsules. The drug or pharmaceutical agent is solubilized in the formulation and has significantly improved bio-availability when compared to the drug tested in its pure form.

### BACKGROUND OF THE INVENTION

1. Field of the Invention

The present invention is in the field of pharmaceutical formulations. More particularly, the present invention pertains to free-flowing solid formulations of drugs, which per se are poorly soluble in water, and where the formulation nevertheless provides improved bio-availability of the drug.

2. Brief Description of Prior Art

Many pharmaceutical agents or drugs are insoluble or have only poor solubility in water. The use of such drugs in a solid form for oral administration, such as tablets or capsules, is hampered by the relatively poor bio-availability of the drug from the solid form. For example, only less than 5% of active drug from brand name drug ZOCOR® reach the general circulation as an active inhibitor. In addition, poorly absorbed drugs often display larger inter- and intra-subject variation in bio-availability. Nevertheless, administration of drugs in a solid form, such as tablets or capsules, is generally preferred over administration of the drug in oral liquid form, or in the form of injections.

Increasing the bio-availability of solid dosage forms posts great challenge to researchers due to either low drug load in most formulations or complexity of the process to prepare the formulations. Limited success in liquid preparations cannot be translated into solid dosages due to the low drug loads that are attained in most solid formulations, which therefore do not offer reasonable therapeutic strength. Thus, a great challenge remains to develop a solid dosage formulation that has a high enough quantity (load) of the insoluble or poorly soluble drugs to provide therapeutic effects as well as enhanced bio-availability.

One approach in the prior art has been to utilize microemulsions, particularly self-microemulsifying drug delivery systems (SMEDDS), to increase the bio-availability of poorly water-soluble drugs. U.S. Pat. Nos. 6,143,321; 6,110,490; 6,309,665; 6,312,704; 5,444,041; 5,993,858; 5,972,911; 5,989,583; 6,337,087; 6,103,259; 6,146,825; 6,337,087; 6,231,882; 6,130,209; 6,120,794; 6,017,545; 6,013,665; 6,248,360; 6,054,136; 6,346,273; 6,027,747; and 6,248,363 are of interest in this regard. A desirable feature of SMEDDS is their ability to form microemulsions when exposed to gastrointestinal fluids.

Microemulsions spontaneously form when precise concentrations of each component are used. A distinguishing feature between emulsion and microemulsion is that the latter is thermodynamically stable and transparent or translucent by itself, compared to a milky appearance of an emulsion which are, generally speaking, thermodynamically unstable and eventually separate. However, oil-in-water microemulsions will become emulsions when diluted with water or aqueous solution because of the lack of appropriate proportions of the components in the system.

U.S. Pat. No. 6,280,770 discloses microemulsion systems as solid dosage forms for oral administration. A microemulsion of the drug is adsorbed onto a solid carrier to form a free-flowing compressible powder that may be further formulated into solid dosage forms such as tablets or capsules. It appears that the concentration of the drugs in the powders (i. e. the drug load) of the solid forms in this reference is significantly lower than the drug load that can be attained in accordance with the present invention.

Micelles have been successfully used in many applications to increase solubility of lipophilic compounds while increasing bio-availability. An appealing feature of micelles over microemulsions is their smaller droplet size (5 nm vs. 20 nm). Due to their smaller size, micelles increase solubility and enhance penetration of the drug. U.S. Pat. No. 4,572,915 discloses a process of micellizing fat-soluble vitamins, essential oils and other fat-soluble agents for liquid preparations in nutritional supplements and cosmetics. Clinical trials with micellized vitamin A and E showed 3-5 times more absorption of these vitamins than those in edible oils. Unlike microemulsions, micellized fat-soluble vitamins can be added to water and result in transparent solutions.

In making micelles, real challenge lies on incorporating sufficient amount of pharmaceutically active agents into formulation. This is especially true when the active agents are in solid form. The present invention provides a solution to the problem of solubilizing and enhancing the bio-availability of poorly soluble drugs and making them available for oral ingestion in a solid form with enhanced drug load when desired.

### SUMMARY OF THE INVENTION

It is an object of the present invention to provide solid formulations of good or improved bio-availability for drugs or pharmaceutical agents which have poor solubility in water.

It is another object of the present invention to provide liquid or gel formulations for drugs or pharmaceutical agents which have poor solubility in water where the liquid or gel is readily absorbable by a suitable solid carrier to provide solid formulations of good or improved bio-availability of the drugs or pharmaceutical agents.

The foregoing and other objects and advantages are attained by first obtaining a liquid or gel formulation that contains the following ingredients or components:

1 to 30 per cent by weight of a pharmaceutical agent or drug that has poor solubility in water;

5 to 60 per cent by weight of a pharmaceutically acceptable surfactant;

10 to 40 per cent by weight of water;

1 to 20 per cent by weight of an unsaturated fatty acid ester;

0 to 50 per cent by weight of a water miscible pharmaceutically acceptable polyol;

1 to 10 per cent by weight of a pharmaceutically acceptable phospholipid;

The liquid or gel formulation of the foregoing composition is readily absorbed by a pharmaceutically acceptable suitable solid carrier such as silicon dioxide, maltodextrin, magnesium oxide, aluminum hydroxide or magnesium trisilicate, or starch to provide a free flowing powder which can be used as such or can be admixed with more and/or other excipients normally used in the pharmaceutical industry to provide tablets, capsules or other solid formulations. Tests indicate that the active drug or pharmaceutical agent has good solubility from the free-flowing solid formulations obtained in the above-described manner in accordance with the invention.

### DETAILED DESCRIPTION OF THE INVENTION, GENERAL EMBODIMENTS AND SPECIFIC EXAMPLES

The following specification sets forth the preferred embodiments of the present invention. The embodiments of the invention disclosed herein are the best modes contemplated by the inventors for carrying out their invention in a commercial environment, although it should be understood that various modifications can be accomplished within the parameters of the present invention.

### General Embodiments

The present invention is utilized to provide first a liquid or gel and thereafter a solid formulation of drugs or pharmaceutical agents where the solid formulation has good or improved bio-availability of the drug or pharmaceutical agent. Those skilled in the art will readily appreciate on the basis of the ensuing description that virtually any drug or pharmaceutical agent can be formulated in accordance with the present invention. Nevertheless, the formulation of the present invention provides a significant improvement or advantage in terms of bio-availability of drugs which have relatively low aqueous solubility and which in prior art solid formulations have less-than-desired bio-availability. For this reason in the ensuing description and in the specific examples reference is made to drugs or pharmaceutical agents of relatively low aqueous solubility.

Generally speaking, many drugs or pharmaceutical agents have lipophilic, character and therefore have low solubility in water. This is especially true of drugs which are not salts and/or do not include a dominant acidic group such as a carboxylic acid or sulfonic acid that would render the drug aqueous soluble at basic or mildly basic pH, nor a mildly basic group, such as an amino group that would render the drug aqueous soluble at acidic or mildly acidic pH. Moreover, there are even drugs which do include a carboxylic acid, sulfonic acid amino or other mildly basic group and nevertheless have poor solubility in aqueous media. Whereas it is difficult to provide a numerical limit as to what is considered poor aqueous solubility for a drug or pharmaceutical agent, a solubility of less than 0.0001 per cent weight by weight would be considered poor or insoluble.

Still speaking generally, many hormones and other drugs containing the steroid skeleton, cholesterol lowering drugs, anti-acids, anti-inflammatory and anti-allergy drugs have low solubility in water and are well suited for the formulation of the present invention that improves their bio-availability. More specific examples of such drugs are: progesterone, lovastatin, simvastatin, famotidine, loratadine, oxametacine, piroxicam, hydrochlorothiazide, acrivastine, estradiol and its esters having estradiol-like activity, norethindrone, estrone and its esters having estrone-like activity, nifedipine, oxymetholone, testosterone and derivatives having testosterone-like activity, carvedilol, chlorthalidone, guanfacine hydrochloride, trandolapril, enalapril maleate, felodipine, amlodipine, colestipol hydrochloride, clofibrate, gemfibrozil, fenofibrate, atorvastatin and pravastatin.

The chemical structures and scientific chemical names of progesterone, lovastatin, simvastatin, famotidine, loratadine oxametacine, piroxicam, hydrochlorothiazide, acrivastine, estradiol and its esters having estradiol-like activity, norethindrone, estrone and its esters having estrone-like activity, nifedipine, oxymetholone, testosterone and derivatives having testosterone-like activity, carvedilol, chlorthalidone, guanfacine hydrochloride, trandolapril, enalapril maleate, felodipine, amlodipine, colestipol hydrochloride, clofibrate, gemfibrozil, fenofibrate, atorvastatin and pravastatin can be found in standard reference books, such as The Merck Index (twelfth edition). It should be remembered that the present invention is not limited by the specific name or chemical structure of the drug or pharmaceutical agent that is incorporated in the formulation.

Another important or principal component of the formulations of the present invention is a pharmaceutically acceptable surfactant or emulsifying agent, examples of which are polyoxyethylene sorbitan fatty acid esters, polyoxyethylene alkyl ethers, polyoxyethylene castor oil derivatives, polyoxyethylene stearates, and saturated polyglycolized glycerides. These pharmaceutically acceptable surfactants are well known in the art and are available from commercial sources.

Specific examples of the surfactants that are used to prepare the preferred embodiments or examples of the present invention are: POE(20) sorbitan monooleate (available under the commercial name Polysorbate 80 Glycosperse O-20); polyoxyl 4-lauryl ether (available under the commercial name Brij 30); polyoxyl 35 castor oil (available under the commercial name as Cremophor EL); lauroyl macrogol-32 glycerides (available under the commercial name as Gelucire 44/14); polyoxyl 50 stearate (available under the commercial name Myrj 53); diethylene glycol monoethyl ether (available under the commercial name Transcutol P).

A function of the surfactant or emulsifying agent is to stabilize in conjunction with the other components and likely in micelles, and thereby solubilize, again in conjunction with the other components, the active drug or pharmaceutical agent of the formulation. As noted above, the drug or pharmaceutical agent used in the formulation is likely to have poor aqueous solubility, and without this solubilization that occurs through micellization, only a significantly lesser amount of the drug could be dissolved in the amount of water used in the formulation, and the increased bio-availability could not be achieved.

The surfactant or emulsifying agent used in the formulation can be a single product, or a combination of two or more of the products or components identified above. Generally speaking, where more than one chemical compound or substance of a certain general category (such as surfactant, unsaturated fatty acid ester, polyol, or phospholipid, preservative or flavoring agent etc.) can be utilized in the present invention, then instead of a single such compound or substance a combination of substances falling within the same general category can also be used.

A further important component of the formulations of the present invention is a pharmaceutically acceptable ester of an unsaturated fatty acid, the preferred example of which is ethyl linoleate. The ester of the unsaturated fatty acid, such as ethyl linoleate, acts as a solubilizing agent. Other examples of suitable unsaturated fatty acids are palmitoleic acid, oleic acid, linoleic acid, which can be present in the composition individually or in combination.

Still another component of the formulations of the present invention is a water miscible and pharmaceutically acceptable polyol, the preferred example of which is propylene glycol. Examples of other suitable water miscible and pharmaceutically acceptable polyols are glycerol, and diethylene glycol, diethylene glycol monoethyl ether (available under the commercial name Transcutol P) and polyethylene glycol. The water miscible, pharmaceutically acceptable polyol acts as an emulsifying or solubilizing agent and also increases the viscosity of the liquid or gel formulations which are first obtained in accordance with the present invention. However, the water miscible and pharmaceutically acceptable polyol is not absolutely essential for preparing the formulations of the present invention, and it is for this reason that its percent range is indicated in the Summary of the Invention as 0 to 50%. Nevertheless, the inclusion of a water miscible and pharmaceutically acceptable polyol in the formulations is preferred and propylene glycol is present in all of the specific examples described below.

Still another important component in the formulation of the invention is comprised of phospholipids. The function of the phospholipids is also to solubilize the drug or pharmaceutical agent. A preferred example of the pharmaceutically acceptable phospholipids included in the formulations of the present invention is lecithin. Other examples of phospholipids suitable for incorporation in the present invention are phosphatidylethanolamine, phosphatidylserine, phosphatidylinositol. The phospholipid, such as lecithin, can be added in aqueous solution, in which case the water of this solution provides some or all of the water utilized to dissolve and solubilize the above listed components to obtain either a gel or a liquid solution.

As noted above, yet another component in the formulation of the present invention is water, which in accordance with practice in the pharmaceutical industry is either de-ionized or distilled.

Preferred ranges of the components in the liquid or gel formulations of the present invention are listed below.

| | | |
|---|---|---|
| Pharmaceutical agent or drug | 2 to 15 | per cent by weight; |
| Surfactant | 20 to 40 | per cent by weight; |
| Water | 15 to 30 | per cent by weight; |
| Unsaturated fatty acid ester | 4 to 10 | per cent by weight; |
| Water miscible polyol | 1 to 30 | per cent by weight; |
| Phospholipid | 1 to 5 | per cent by weight. |

The above-noted components are thoroughly admixed in accordance with a General Procedure described below, to provide a clear liquid or clear gel. Those skilled in the art will readily understand that the nature and consistency of the formulation obtained in this manner (whether it is a liquid or gel, and the consistency of the gel) depend on the nature and amounts of the several components used. It should also be understood in connection with the herein listed ranges of percentages of the components, that it is not contemplated within the scope of the invention to have all or most of the ingredients present in their respective maximum listed range in any given composition, as such a composition would be incapable of existence for having more than 100% of the sum of its components. Rather, it is contemplated that when one or more ingredients are in their maximum range, then the ratios of other components are in less than their maximum range, so that the sum total of all components (listed or not listed above) is 100%.

### General Procedure for Admixing Components to Form a Clear Gel or Clear Liquid

The surfactant or emulsifying agent is heated to attain a temperature in the range of 100° C. to 130° C., preferably to approximately 120° C. Then the active drug is slowly added to the surfactant with vigorous stirring until a homogenous, clear solution is obtained. Slowly and consecutively, the water miscible polyol (preferably propylene glycol) and the unsaturated fatty acid (preferably ethyl linoleate) are added in this order. Thereafter, the aqueous solution of the phospholipid (preferably 5% aqueous lecithin solution) is added to the composition with vigorous stirring, to make 100%. The mixture is then cooled immediately in a cold-water bath. After cooling the resulting gel is clear and homogeneous and miscible with water to form a clear solution.

Tests have shown that when gels obtained in accordance with the present invention and more specifically described below in connection with the specific examples are placed in a gastric medium (pH 1.2) then a significant percentage of the drug (simvastatin) is dissolved within minutes. By comparison, when the same drug per se is exposed to gastric medium no measurable amount of the drug is dissolved in comparable time.

The gel or liquid of the formulations which are obtained as described above are suitable as such for administration to mammals, including humans, as a carrier of the drug or pharmaceutical agent contained therein. However, it is preferred in accordance with the present invention that the gel or liquid be absorbed on a suitable pharmaceutically acceptable solid carrier, such as silicon dioxide, maltodextrin, magnesium oxide, aluminum hydroxide, magnesium trisilicate or starch. Among these solid carriers silicon dioxide, particularly colloidal silicon dioxide is presently preferred. These carriers per se well known in the art, and need not be described here further. The gel or liquid formulation can be absorbed by the solid carrier either by granulation or by spray drying. Both the granulation and spray drying processes are well known in the art, and need not be described here further. The liquid or gel formulations absorbed in this manner on the solid carrier become free-flowing powders that are suitable as such for being formed into tablets or capsules. However, other pharmaceutically acceptable excipients can also be added to the free-flowing powder obtained in the above-described manner to make tablets or capsules or other solid form suitable for practical oral administration. In addition, coloring agents, flavoring agents or preservatives and other pharmaceutically acceptable substances that are normally or occasionally included in tablets or capsules in addition to the pharmacologically active drug, can also be included in the tablets or capsules. Such non-active components may, also be added to the formulation while it is a liquid or gel, or before the components are admixed to form a liquid or gel.

Generally speaking, the free flowing powder obtained from the gel or liquid includes 20 to 80 per cent by weight of the gel or liquid and 20 to 80 per cent by weight of the solid carrier. More preferably, the free flowing powder obtained from the gel or liquid includes 50 to 80 per cent by weight of the gel or liquid and 20 to 50 per cent by weight of the solid carrier. Tablets or capsules made by utilizing the free flowing powder may contain the same percentages, or may be further diluted by other excipients, such as microcrystalline cellulose, dicalcium phosphate, stearic acid and magnesium stearate.

### General Procedure for Tableting

Weigh and screen all items except magnesium stearate into a V-blender and blend for approximately 15 minutes. Add magnesium stearate into the blender and mix an additional 5 minutes. Discharge the powder and compress tablets.

### General Procedure for Testing of Tablets

The tablets obtained in accordance with the foregoing procedure are tested on a USP dissolution apparatus with paddles. Dissolution medium is 0.1 N HCl solution at 37° C. Samples are collected and assayed using HPLC.

### SPECIFIC EXAMPLES

### Example 1

| | |
|---|---|
| POE(20) sorbitan monooleate | 34% |
| (Polysorbate 80 Glycosperse O-20) | |
| Propylene Glycol | 24% |
| Ethyl Linoleate | 8% |
| Simvastatin | 10% |
| 5% Lecithin aqueous solution | QS |

The surfactant sorbitan monooleate is heated to 120° C. Simvastatin is slowly added to the surfactant with vigorous stirring until a homogenous, clear solution is obtained. Slowly and consecutively, propylene glycol and ethyl linoleate are added. Then 5% aqueous lecithin solution is added to the composition with vigorous stirring, to make 100%. The resulting clear gel is immediately cooled in a cold-water bath. The cooled gel is clear and homogeneous and miscible with water to form a clear solution. Tests of in vitro dissolution in a gastric medium at pH 1.2 showed that 24% of simvastatin dissolved within 10 minutes of exposure to the gastric medium.

### Example 2

| | |
|---|---|
| POE(20) sorbitan monooleate | 35% |
| (Polysorbate 80 Glycosperse O-20) | |
| Propylene Glycol | 25% |
| Ethyl Linoleate | 8% |
| Simvastatin | 4% |
| 5% Lecithin aqueous solution | QS |

The above components are admixed as described in the General Procedure to provide a clear gel at room temperature. This gel is miscible with water to form a clear solution. When the gel was agitated in a gastric medium of pH 1.2 74% of the drug simvastatin dissolved within 10 minutes. By comparison, when the drug simvastatin per se was exposed to the same medium no measurable amount of the drug could be detected in solution after exposure of comparable time.

The term QS in this and in the other specific examples means that sufficient 5% aqueous lecithin solution is added to the composition to make 100 per cent. The lecithin solution in this example is 5 percent weight by weight. Thus, if one were to make a 100 grams total of the formulation of Example 2, then 28 grams of 5% aqueous solution would be combined with the other components. 28 grams of 5% aqueous lecithin solution contains 1.4 lecithin (phospholipid) and 26.6 grams of water.

### Example 3

| | |
|---|---|
| polyoxyl 4-lauryl ether (Brij 30) | 35% |
| Propylene Glycol | 25% |
| Ethyl Linoleate | 8% |
| Simvastatin | 4% |
| 5% Lecithin aqueous solution | QS |

The above components are admixed as described in the General Procedure to provide a clear gel at room temperature. When the gel was exposed to a gastric medium of pH 1.2, 52% of the drug simvastatin dissolved within 10 minutes.

### Example 4

| | |
|---|---|
| polyoxyl 35 castor oil (Cremophore EL) | 35% |
| Propylene Glycol | 25% |
| Ethyl Linoleate | 8% |
| Simvastatin | 4% |
| 5% Lecithin aqueous solution | QS |

The above components are admixed as described in the General Procedure to provide a clear gel at room temperature. When the gel was exposed to a gastric medium of pH 1.2, 52 % of the drug simvastatin dissolved within 10 minutes.

### Example 5

| | |
|---|---|
| polyoxyl 35 castor oil (Cremophore EL) | 35% |
| Propylene Glycol | 25% |
| Ethyl Linoleate | 8% |
| Simvastatin | 8% |
| 5% Lecithin aqueous solution | QS |

The above components are admixed as described in the General Procedure to provide a clear gel at room temperature. When the gel was exposed to a gastric medium of pH 1.2, 37 % of the drug simvastatin dissolved within 10 minutes.

### Example 6

| | |
|---|---|
| lauroyl macrogol-32 glycerides (Gelucire 44/14) | 35% |
| Propylene Glycol | 25% |
| Ethyl Linoleate | 8% |
| Sunvastatin | 4% |
| 5% Lecithin aqueous solution | QS |

The above components are admixed as described in the General Procedure to provide a clear gel at elevated temperature (>50° C.). When the gel was exposed to a gastric medium of pH 1.2 57 % of the drug simvastatin dissolved within 10 minutes.

### Example 7

| | |
|---|---|
| polyoxyl 50 stearate (Myrj 53) | 35% |
| Propylene Glycol | 25% |
| Ethyl Linoleate | 8% |
| Simvastatin | 4% |
| 5% Lecithin aqueous solution | QS |

The above components are admixed as described in the General Procedure to provide a clear gel at room temperature. When the gel was exposed to a gastric medium of pH 1.2, 65 % of the drug simvastatin dissolved within 10 minutes.

### Example 8

| | |
|---|---|
| POE(20) sorbitan monooleate | 35% |
| (Polysorbate 80 Glycosperse O-20) | |
| Propylene Glycol | 25% |
| Ethyl Linoleate | 8% |
| Diethylene glycol monoethyl ether (Transcutol P) | 6% |
| Simvastatin | 8% |
| 5% Lecithin aqueous solution | QS |

The above components are admixed as described in the General Procedure to provide a clear gel at room temperature. When the gel was exposed to a gastric medium of pH 1.2, 57 % of the drug simvastatin dissolved within 10 minutes.

### Example 9

| | |
|---|---|
| POE(20) sorbitan monooleate | 35% |
| (Polysorbate 80 Glycosperse O-20) | |
| Polyethylene Glycol | 25% |
| Ethyl Linoleate | 8% |
| Simvastatin | 4% |
| 5% Lecithin aqueous solution | QS |

The above components are admixed as described in the General Procedure to provide a clear gel at room temperature. When the gel was exposed to a gastric medium of pH 1.2, 69 % of the drug simvastatin dissolved within 10 minutes.

### Preparation of Free-Flowing Powder

### Example 10

30% of colloidal silicon dioxide is granulated with 70% of the gel prepared in Example 2 to yield a uniform wet granulation. The granule is dried at approximately 60 to 80° C. to provide a free-flowing powder. When this powder was exposed to a gastric medium of pH 1.2 67% of the drug simvastatin dissolved within 10 minutes.

### Example 11

30% of colloidal silicon dioxide is granulated with 70% of the gel prepared in Example 3 to yield a uniform wet granulation. The granule is dried at approximately 60 to 80° C. to provide a free-flowing powder. When this powder was exposed to a gastric medium of pH 1.2, 52% of the drug simvastatin dissolved within 10 minutes.

### Example 12

30% of colloidal silicon dioxide is granulated with 70% of the gel prepared in Example 4 to yield a uniform wet granulation. The granule is dried at approximately 60 to 80° C. temperature to provide a free-flowing powder. When this powder was exposed to a gastric medium of pH 1.2, 52% of the drug simvastatin dissolved within 10 minutes.

### Example 13

30% of colloidal silicon dioxide is granulated with 70% of the gel prepared in Example 8 to yield a uniform wet granulation. The granule is dried at approximately 60 to 80° C. to provide a free-flowing powder. When this powder was exposed to a gastric medium of pH 1.2, 57% of the drug simvastatin dissolved within 10 minutes.

The amount of drug dissolved in gastric medium from the free flowing powders of Examples 10, 11, 12 and 13 is the same, or closely the same as the amount dissolved in similar tests from the corresponding gel formulations.

### Preparation of Tablets

### Example 14

Ingredients for a 550 mg tablet

| | | |
|---|---|---|
| Free flowing powder from Example 8 | 145 mg | 26.36% |
| (6.9% Simvastatin by HPLC Assay) | | |
| Microcrystalline Cellulose | 235 mg | 42.72% |
| Dicalcium Phosphate | 135 mg | 24.53% |
| Stearic Acid | 30 mg | 5.45% |
| Magnesium Stearate | 5 mg | 0.91% |

The tablets are prepared as described in the general procedure for making tablets. When tested in a USP dissolution apparatus with paddles with a medium of 0.1 N HCl solution at 37° C. Samples are collected and assayed using HPLC. Within 30 minute, 50% label-claimed of simvastatin is detected, compared to undetectable amount of simvastatin when the drug is not micellized.

What is claimed is:

1. A composition comprising:
1 to 30 per cent by weight of a pharmaceutical agent or drug that has poor solubility in water;
5 to 60 per cent by weight of a pharmaceutically acceptable surfactant;
10 to 40 per cent by weight of water;
1 to 20 per cent by weight of a pharmaceutically acceptable unsaturated fatty acid ester;
0 to 50 per cent by weight of a pharmaceutically acceptable water miscible polyol, and
1 to 10 per cent by weight of a pharmaceutically acceptable phospholipid.

2. A composition in accordance with claim 1 that is a gel.

3. A composition in accordance with claim 1 that is a liquid.

4. A composition in accordance with claim 1 where the drug has less than 0.0001 per cent weight by weight solubility in water.

5. A composition in accordance with claim 1 comprising:

| | |
|---|---|
| pharmaceutical agent or drug | 2 to 15 percent by weight; |
| surfactant | 20 to 40 percent by weight; |
| water | 15 to 30 percent by weight; |
| unsaturated fatty acid ester | 4 to 10 percent by weight; |
| water miscible polyol | 1 to 30 percent by weight; |
| phospholipid | 1 to 5 percent by weight. |

6. A composition in accordance with claim 4 comprising:

| | |
|---|---|
| pharmaceutical agent or drug | 2 to 15 percent by weight; |
| surfactant | 20 to 40 percent by weight; |
| water | 15 to 30 percent by weight; |
| unsaturated fatty acid ester | 4 to 10 percent by weight; |
| water miscible polyol | 1 to 30 percent by weight; |
| phospholipid | 1 to 5 percent by weight. |

7. A composition in accordance with claim 1 where the drug is selected from the group consisting of hormones, cholesterol lowering drugs, anti-acids and anti-allergy drugs.

8. A composition in accordance with claim 1 where the drug is selected from the group consisting of progesterone, lovastatin, simvastatin, famotidine, loratadine, oxametacine, piroxicam, hydrochlorothiazide, acrivastine, estradiol and its esters having estradiol-like activity, norethindrone, estrone and its esters having estrone-like activity, nifedipine, oxymetholone, testosterone and derivatives having testosterone-like activity, carvedilol, chlorthalidone, guanfacine hydrochloride, trandolapril, enalapril maleate, felodipine, amlodipine, colestipol hydrochloride, clofibrate, gemfibrozil, fenofibrate, atorvastatin and pravastatin.

9. A composition in accordance with claim 1 where the surfactant is selected from the group consisting of polyoxyethylene sorbitan fatty acid esters, polyoxyethylene alkyl ethers, polyoxyethylene castor oil derivatives, polyoxyethylene stearates, and saturated polyglycolized glycerides.

10. A composition in accordance with claim 1 where the unsaturated fatty acid is selected from the group consisting of ethyl linoleate, palmitoleic acid, oleic acid and linoleic acid.

11. A composition in accordance with claim 1 where the water miscible polyol is selected from the group consisting of propylene glycol, glycerol, diethylene glycol, diethylene glycol monoethyl ether and polyethylene glycol.

12. A composition in accordance with claim 1 where the phospholipid is selected from the group consisting of lecithin, phosphatidylethanolamine, phosphatidylserine and phosphatidylinositol.

13. A composition comprising:
1 to 30 per cent by weight of a pharmaceutical agent or drug that has poor solubility in water;
5 to 60 per cent by weight of a pharmaceutically acceptable surfactant selected from the groups consisting of polyoxyethylene sorbitan fatty acid esters, polyoxyethylene alkyl ethers, polyoxyethylene castor oil derivatives, polyoxyethylene stearates, and saturated polyglycolized glycerides;
10 to 40 per cent by weight of water;
1 to 20 per cent by weight of a pharmaceutically acceptable unsaturated fatty acid ester selected from the groups consisting of ethyl linoleate, palmitoleic acid, oleic acid and linoleic acid;
0 to 50 per cent by weight of a pharmaceutically acceptable water miscible polyol selected from the group consisting of propylene glycol, glycerol, diethylene glycol, diethylene glycol monoethyl ether and polyethylene glycol, and
1 to 10 per cent by weight of a pharmaceutically acceptable phospholipid selected from the group consisting of lecithin, phosphatidylethanolamine, phosphatidylserine and phosphatidylinositol.

14. A composition in accordance with claim 13 where the drug has less than 0.0001 per cent weight by weight solubility in water.

15. A composition in accordance with claim 14 comprising:

| | |
|---|---|
| pharmaceutical agent or drug | 2 to 15 percent by weight; |
| surfactant | 20 to 40 percent by weight; |
| water | 15 to 30 percent by weight; |
| unsaturated fatty acid ester | 4 to 10 percent by weight; |
| water miscible polyol | 1 to 30 percent by weight; |
| phospholipid | 1 to 5 percent by weight. |

16. A composition in accordance with claim 13 where the drug is selected from the group consisting of hormones, cholesterol lowering drugs, anti-acids and anti-allergy drugs.

17. A composition in accordance with claim 13 where the drug is selected from the group consisting of progesterone, lovastatin, simvastatin, famotidine, loratadine, oxametacine, piroxicam, hydrochlorothiazide, acrivastine, estradiol and its esters having estradiol-like activity, norethindrone, estrone and its esters having estrone-like activity, nifedipine, oxymetholone, testosterone and derivatives having testosterone-like activity, carvedilol, chlorthalidone, guanfacine hydrochloride, trandolapril, enalapril maleate, felodipine, amlodipine, colestipol hydrochloride, clofibrate, gemfibrozil, fenofibrate, atorvastatin and pravastatin.

18. A solid formulation of a pharmaceutical agent or drug that is obtained by a process comprising the steps of admixing a liquid or gel composition that comprises
1 to 30 per cent by weight of a pharmaceutical agent or drug that has poor solubility in water;
5 to 60 per cent by weight of a pharmaceutically acceptable surfactant;
10 to 40 per cent by weight of water;
1 to 20 per cent by weight of a pharmaceutically acceptable unsaturated fatty acid ester;
0 to 50 per cent by weight of a pharmaceutically acceptable water miscible polyol, and
1 to 10 per cent by weight of a pharmaceutically acceptable phospholipid with a pharmaceutically acceptable solid carrier and thereafter drying the admixture to obtain a free-flowing powder.

19. A solid formulation in accordance with claim 18 wherein the process of obtaining the formulation further includes the step of forming the free-flowing powder into tablets or capsules.

20. A solid formulation in accordance with claim 18 where the pharmaceutically acceptable solid carrier is selected from the group consisting of silicon dioxide, maltodextrin, magnesium oxide, aluminum hydroxide, magnesium trisilicate and starch.

21. A solid formulation, in accordance with claim 18 where the pharmaceutically acceptable solid carrier is colloidal silicon dioxide.

22. A solid formulation in accordance with claim 18 where the liquid or gel composition used in the process of admixing comprises

| | |
|---|---|
| pharmaceutical agent or drug | 2 to 15 percent by weight; |
| surfactant | 20 to 40 percent by weight; |
| water | 15 to 30 percent by weight; |
| unsaturated fatty acid ester | 4 to 10 percent by weight; |
| water miscible polyol | 1 to 30 percent by weight; |
| phospholipid | 1 to 5 percent by weight. |

23. A solid formulation in accordance with claim 18 where the drug has less than 0.0001 per cent weight by weight solubility in water.

24. A solid formulation in accordance with claim 23 where the drug has less than 0.0001 per cent weight by weight solubility in water.

25. A solid formulation in accordance with claim 18 where the drug is selected from the group consisting of hormones, cholesterol lowering drugs, anti-acids and anti-allergy drugs.

26. A solid formulation in accordance with claim 18 where the drug is selected from the group consisting of progesterone, lovastatin, simvastatin, famotidine, loratadine, oxametacine, piroxicam, hydrochlorothiazide, acrivastine, estradiol and its esters having estradiol-like activity, norethindrone, estrone and its esters having estrone-like activity, nifedipine, oxymetholone, testosterone and derivatives having testosterone-like activity, carvedilol, chlorthalidone, guanfacine hydrochloride, trandolapril, enalapril maleate, felodipine, amlodipine, colestipol hydrochloride, clofibrate, gemfibrozil, fenofibrate, atorvastatin and pravastatin.

27. A solid formulation in accordance with claim 18 where the surfactant is selected from the group consisting of polyoxyethylene sorbitan fatty acid esters, polyoxyethylene alkyl ethers, polyoxyethylene castor oil derivatives, polyoxyethylene stearates, and saturated polyglycolized glycerides.

28. A solid formulation in accordance with claim 18 where the unsaturated fatty acid is selected from the group consisting of ethyl linoleate, palmitoleic acid, oleic acid and linoleic acid.

29. A solid formulation in accordance with claim 18 where the water miscible polyol is selected from the group consisting of propylene glycol, glycerol, diethylene glycol, diethylene glycol monoethyl ether and polyethylene glycol.

30. A solid formulation in accordance with claim 18 where the phospholipid is selected from the group consisting of lecithin, phosphatidylethanolamine, phosphatidylserine and phosphatidylinositol.

31. A solid formulation of a pharmaceutical agent or drug that is obtained by a process comprising the steps of admixing a liquid or gel composition that comprises
1 to 30 per cent by weight of a pharmaceutical agent or drug that has poor solubility in water;
5 to 60 per cent by weight of a pharmaceutically acceptable surfactant selected from the groups consisting of polyoxyethylene sorbitan fatty acid esters, polyoxyethylene alkyl ethers, polyoxyethylene castor oil derivatives, polyoxyethylene stearates, and saturated polyglycolized glycerides;
10 to 40 per cent by weight of water;
1 to 20 per cent by weight of a pharmaceutically acceptable unsaturated fatty acid ester selected from the groups consisting of ethyl linoleate and palmitoleic acid, oleic acid and linoleic acid;
0 to 50 per cent by weight of a pharmaceutically acceptable water miscible polyol selected from the group consisting of propylene glycol, glycerol, diethylene glycol, diethylene glycol monoethyl ether and polyethylene glycol, and
1 to 10 per cent by weight of a pharmaceutically acceptable phospholipid selected from the group consisting of lecithin with a pharmaceutically acceptable solid carrier and thereafter drying the admixture to obtain a free-flowing powder.

32. A solid formulation in accordance with claim 31 wherein the process of obtaining the formulation further includes the step of forming the free-flowing powder into tablets or capsules.

33. A solid formulation in accordance with claim 31 where the drug has less than 0.0001 per cent weight by weight solubility in water.

34. A solid formulation in accordance with claim 31 comprising:

| | |
|---|---|
| pharmaceutical agent or drug | 2 to 15 percent by weight; |
| surfactant | 20 to 40 percent by weight; |
| water | 15 to 30 percent by weight; |
| unsaturated fatty acid ester | 4 to 10 percent by weight; |
| water miscible polyol | 1 to 30 percent by weight; |
| phospholipid | 1 to 5 percent by weight. |

35. A solid formulation in accordance with claim 34 where the drug is selected from the group consisting of hormones, cholesterol lowering drugs, anti-acids and anti-allergy drugs.

36. A solid formulation in accordance with claim 34where the drug is selected from the group consisting of progesterone, lovastatin, simvastatin, famotidine, loratadine, oxametacine, piroxicam, hydrochlorothiazide, acrivastine, estradiol and its esters having estradiol-like activity, norethindrone, estrone and its esters having estrone-like activity, nifedipine, oxymetholone, testosterone and derivatives having testosterone-like activity, carvedilol, chlorthalidone, guanfacine hydrochloride, trandolapril, enalapril maleate, felodipine, amlodipine, colestipol hydrochloride, clofibrate, gemfibrozil, fenofibrate, atorvastatin and pravastatin.

37. A solid formulation in accordance with claim 31 where the pharmaceutically acceptable solid carrier is selected from the group consisting of silicon dioxide, maltodextrin, magnesium oxide, aluminum hydroxide, magnesium trisilicate and starch.

38. A solid formulation in accordance with claim 37 where the pharmaceutically acceptable solid carrier is colloidal silicon dioxide.

39. A process for preparing a composition including:
1 to 30 per cent by weight of a pharmaceutical agent or drug that has poor solubility in water;
5 to 60 per cent by weight of a pharmaceutically acceptable surfactant;
10 to 40 per cent by weight of water;
1 to 20 per cent by weight of a pharmaceutically acceptable unsaturated fatty acid ester;
0 to 50 per cent by weight of a pharmaceutically acceptable water miscible polyol, and
1 to 10 per cent by weight of a pharmaceutically acceptable phospholipid,
the process comprising the steps of:
having the surfactant in the temperature range of 100° C. to 130° C.;
adding the pharmaceutical agent or drug with stirring until a homogenous, clear solution is obtained;
thereafter adding the water miscible polyol and adding the unsaturated fatty acid ester;
thereafter adding with stirring the phospholipid dissolved in water, and
thereafter cooling the admixture to room temperature to provide a clear homogeneous composition.

40. A process in accordance with claim 39 further comprising the step of admixing the cooled clear homogeneous composition with a pharmaceutically acceptable solid carrier selected from the group consisting of silicon dioxide, maltodextrin, magnesium oxide, aluminum hydroxide, magnesium trisilicate and starch and thereafter drying said admixture to yield a free flowing powder.

41. A process in accordance with claim 40 further comprising the step to adding one or more pharmaceutically acceptable excipient to said free flowing powder and compressing the admixture into tablets or capsules.
